(19) United States
(12) Patent Application Publication
(10) Pub. No.: US 2026/0023087 A1
(43) Pub. Date: Jan. 22, 2026

(54) A Novel Chemogenetic Tool Permitting Modulation of Intracellular pH

(71) Applicants: ISTANBUL MEDIPOL UNIVERSITESI, ISTANBUL (TR); SABANCI UNIVERSITESI, Tuzla/Istanbul (TR)

(72) Inventors: Asal Ghaffari Zaki, Pendik/Istanbul (TR); Seyed Mohammad Miri, Pendik/Istanbul (TR); Gurkan Ozturk, Uskudar/Istanbul (TR); Emrah Eroglu, Maltepe/Istanbul (TR)

(21) Appl. No.: 19/145,493

(22) PCT Filed: Jan. 5, 2024
(86) PCT No.: PCT/TR2024/050007

§ 371 Date: Jul. 2, 2025

(30) Foreign Application Priority Data
Jan. 6, 2023 (TR) 2023/000206
Mar. 15, 2023 (TR) 2023/002881

(51) Int. Cl. G01N 33/84; C12N 15/85; C12Q 1/527; G01N 21/64

(52) U.S. Cl. CPC G01N 33/84; C12N 15/85; C12Q 1/527; G01N 21/6428; G01N 2333/988

(57) ABSTRACT

The invention relates to a new chemogenetic tool manipulating intracellular pH levels and hydrogen sulfide levels in living cells and tissues with high spatio-temporal resolution.

### TECHNICAL FIELD OF THE INVENTION

A new method is presented that permits manipulating intracellular pH levels and hydrogen sulfide levels in living cells and tissues with high spatio-temporal resolution. This approach is a substrate-based chemogenetic approach, which permits the biochemical conversion of β-Chloro D-alanine to hydrochloric acid or D-cysteine to hydrogen sulfide, respectively, when the substrates are provided to the enzyme. This invention presents a novel tool to study pH-/or hydrogen sulfide-dependent signaling and metabolic pathways under normal and disease conditions.

### STATE OF THE ART OF THE INVENTION (PRIOR ART)

Intracellular pH levels are tightly regulated. Gene expression, cell motility, and metabolic processes are a few examples of the many cellular processes under the control of local pH fluctuations. Hence, multiple disorders, such as cancer, cardiovascular disease, and neurological diseases, may be associated with the dysregulation of pH. The ability to monitor and manipulate intracellular pH levels directly inside a single cell has enormous ramifications for understanding subcellular and suborganelle processes, disease diagnosis, and developing novel therapeutic strategies. Several technologies have been advanced to investigate the role of pH at single cell level; however, conventional methods such as the application of micropipettes, genetic or chemical manipulation of proton pumps, optogenetic approaches, and small chemical inhibitors have off-target effects or are less practical. Therefore, the lack of tractable experimental tools permitting manipulating pH levels with high spatio-temporal resolution in the acidic range undermines studying the relationship between pH imbalance and cell function in health and disease.

Previous attempts to manipulate internal pH (pHi) levels include the overexpression or disruption of ion transporters, exogenous administration of drugs (e.g., cariporide, EIPA, DMA, or amiloride) and using micropipettes. Very recently, an optogenetic tool has been developed to address the lack of an appropriate tool for the regulation of pHi precisely and specifically. Optogenetics and chemogenetics are very sophisticated tools that permit the modulation of physiological events at a single-cell level. In the optogenetic approach, authors employed a light-driven outward proton pump, Archaerhodopsin, to increase pHi in a physiological range. Using this method, they could show the relationship between the rise in pHi and membrane ruffling and dynamics. However, this sophisticated tool can only elicit an increase in pHi. It cannot cover the acidic pHi, which is even more critical in cell physiology and behavior, as some disease conditions are associated with a drop in pHi, such as neurodegeneration. Moreover, while optogenetic tools can provide high spatiotemporal resolution, chemogenetic tools permit controlled manipulation in very deep tissues and specific ultra-locales (subcellular locales such as mitochondria, nucleus, cytosol, ER, Golgi, caveolae, etc.). Importantly, optogenetic approaches need state-of-the-art and expensive facilities and a high level of expertise, limiting their widespread use in basic and translational research. A similar situation also applies to H₂S. So far, there is no way to precisely modulate H₂S in single cells. The current solutions to study the role of H₂S in cells are to either knockout/overexpress the endogenous enzymes (such as mercaptopyruvate sulfur transferase, Cystathionine beta-synthase, and Cystathionine gamma-lyase) or provision of H₂S-releasing drugs such as NaHS.

Intracellular pH (pHi) levels and reductive stress are two critical parameters meticulously regulated in the human body. Yet the exact mechanisms are less explored due to the lack of informative technologies. Genetically encoded biosensors developed in the last 20 years have significantly advanced our understanding of these complex biochemical pathways because these tools allow real-time monitoring of these parameters in live cells. However, there are no experimentally tractable approaches that permit the manipulation and perturbation of pH or H₂S levels in cells and tissues endogenously and in a controlled manner to investigate the underlying mechanisms associated with the development of various pathologies. The available methods to manipulate the pHi and H₂S are thoroughly outmoded and do not align with the present scientific era. In this regard, a recent article explored how alteration in transient pHi affects cardiac contraction using NH₄Cl and CH₃COONa perfusion to increase or decrease the cardiac pHi, a non-physiological method developed in 1981. In another study, the specificity of a pH fluorescent probe was evaluated through intracellular acidification by exposing the mouse models to 10% CO2, an uncontrolled and radical approach. In the absence of a robust and specific tool for the manipulation of pHi and H₂S at the single-cell level, studying the role of pHi in cellular physiology and physiopathology is whether impossible or unreliable.

### BRIEF DESCRIPTION AND OBJECTS OF THE INVENTION

The invention allows to manipulate pH and H₂S levels at ultra-local levels in cells and tissues selectively and with ultra-precision to understand molecular mechanisms. This multifunctional chemogenetic approach is based on a previously described enzyme termed stDCyD (D-cysteine desulfhydrase) derived from Salmonella typhimurium. This technology can be used to develop a novel animal model system to mimic different pathologies such as (but not limited to) cardiovascular dysfunction, neurological disorders, immunological pathologies, cancer, and metabolic diseases. Also, this approach is potentially helpful for the treatment of diseases or the development of therapeutic strategies.

The mentioned chemogenetic tool can be expressed in specific tissues, cell types, and subcellular locales, which permits precisely manipulating the internal pH (pHi) and H₂S levels with high spatio-temporal resolution. These manipulations can be done on a single cell and in vivo. Since the mentioned tool is genetically encoded, it can be fused to any fluorescent protein, permitting real-time detection of the enzyme. Additionally, stDCyD can generate HCl and H₂S in a dose-dependent manner (Figure le), which means that depending on the biological question, the concentration of the substrate can be optimized. Another advantage of approach is that stDCyD remains silent without its substrates (βCDA or D-cysteine). Thus, the chemogenetic approach no longer produces HCl or H₂S upon removing these substrates from cells. Besides, this chemogenetic approach does not have unspecific effects as its substrates do not exist in mammalian cells (except D-cysteine, which can exist in a few tissues such as the liver and brain), and the amount of byproducts of the enzymatic reaction is negligible (Table 1, FIG. 4).

Significant fields of application of such a novel chemogenetic approach can be:
i.) Basic research to investigate the role of intracellular pH and H₂S levels, both relevant for a plethora of pathologies ranging from cardiovascular diseases to neurodegenerative diseases and the core cellular biochemistry.
ii.) For the development of a novel animal model system that may serve for investigating the efficacy of different drugs related to altered pH levels or hydrogen sulfide signaling. These models can also mimic relevant phenotypic changes in health and disease and find therapeutic targets for distinct pathological conditions.
iii.) Currently, the incorporation of proton transfer inhibitors and other cellular acidifiers is being developed for cancer treatment. Therefore, our chemogenetic tools may be exploitable for therapeutic strategies as a recombinant protein manipulating pH or H₂S levels in tissues as an alternative to conventional therapeutic approaches.

This approach permits:
i) Manipulation of intracellular pH levels is a critical feature because it is nearly impossible with currently available conventional approaches to manipulate and investigate the role of pH levels in subcellular locales such as the mitochondria, nucleus, cytosol, lysosomes, and endoplasmic reticulum. Our suggested approach can address this deficiency as it is a genetically encoded enzyme that can be fused to targeting sequences and be specifically expressed and localized in the desired compartments within the cell of interest.
ii) Some of the available methods for manipulating pHi and H₂S levels in cells employ the inhibition of ion transporters using chemicals. For example, inhibitors of NHE1 and V-ATPase have been used to decrease pHi; however, they have shown some off-target effects due to the nature of such transporters as they exchange protons at the expense of other molecules and ions; thereby, these methods lack the required specificity needed to study the role of pHi under physiological settings. On the other hand, our chemogenetic approach has low to zero unspecific responses. The byproducts of stDCyD enzymatic activity are pyruvate and ammonium, and the increase in their concentrations falls in the physiologically acceptable range.

### Definitions of Figures Describing the Invention

FIG. 1. Characterization of pH-Control. a) Schematic representation of the pH-Control pathway and its simultaneous visualization with the pH-sensitive biosensor SypHer3s. b) Representative confocal images of HEK293T cells co-expressing DsRed-stDCyD. Scale bar=20 μm. c) Real-time SypHer3s traces of cytosolic pH in WT cells (n= 3/39) or cells expressing DsRed-stDCyD (n= 3/32) in response to 13.4 mM βCDA. d) Bars show SypHer3s biosensor responses in cells expressing the WT DsRed-stDCyD (n= 4/21) and mutated and nonfunctional DsRed-stDCyD (n= 4/18) upon administration of 1 mM βCDA. e) Left panel shows a representative curve of SypHer3s in response to various concentrations of βCDA as indicated in the figure. The right panel shows a concentration-response curve in HEK293T cells without enzyme (green curve) or expressing stDCyD (red curve) upon administration of indicated concentrations of βCDA. N=3 for all experiments and n=8-49 individual cells. f) Bars show the selectivity test of cells treated with βCDA (n= 3/29) or βCLA (n= 3/29). g) Representative real-time traces of HEK293T cells co-expressing SypHer3s and pH-Control in response imaging medium with different pH levels and 1 mM βCDA as indicated (n= 3/17). h) Representative real-time traces of SypHer signals in DRG neurons expressing pH-Control in response to 10 or 1 mM βCDA. The inset shows representative confocal images of DRG neurons co-expressing SypHer3s and pH-Control. i) Representative confocal images of dorsal root ganglion neurons co-expressing ASAP2s and pH-Control eight days after viral infection. Scale bar=50 μm. j) Representative real-time curve shows signals of the voltage sensor ASAP2s in DRG neurons co-expressing pH-Control in response to high potassium (50 mM) and 10 mM βCDA as indicated (similar results were obtained from 4 different experiments and eleven individual cells). Student's t-test was applied.

FIG. 2. The mutant enzyme remaining inactive in the presence of βDCA in living cells.

FIG. 3. In the presence of a suitable biosensor, i.e., hsGFP, the generation of H₂S and in real-time detection with optical devices.

FIG. 4. Visualizing intracellular pyruvate, a byproduct of the pH-Control activity. (Left) Real-time traces of the FRET-biosensor Pyronic in response to 1 mM βCDA and 1 mM pyruvate. (Right) Bars represent the maximum FRET response upon administration of extracellular pyruvate (n= 5/34) and βCDA (n= 5/34). Student's t-test has been applied.

FIG. 5. Manipulating subcellular pH levels. a) Real-time traces of SypHer biosensor in three different cellular locales, including the cytosol (n= 3/16), mitochondria (n= 3/22), and nucleus (n= 3/33) in response to 1 mM βCDA. b) Representative confocal images show the correct localization of the pH-sensitive biosensor SypHer3s (upper panel) and the pH-Control constructs (middle panel). Lower panels show merged fluorescence images with bright field images.

FIG. 6. Calibration of the pH biosensor SypHer3s. a) Scatter dot blot represents the normalized ratio values of SypHer3s upon titration of extracellular imaging medium with distinct pH levels. Cells have been titrated in the presence of 10 μM monensin and 10 μM nigericin to permeabilize cells. b) Representative real-time traces of HEK293T cells expressing SypHer3s biosensor treated with monensin and nigericin and imaging media with distinct pH levels as indicated in the figure (n= 3/15).

FIG. 7. Overcorrection of intracellular pH levels following acute acidification. (left) Real-time traces of HEK293T cells expressing SypHer and pH-Control in response to 1 mM βCDA in the presence (pink curve and (right) bars, n= 3/15) and absence (green curve and (right) bars n= 3/13) 10 μM nigericin and 10 μM monensin that permeabilize the plasma membrane. Student's t-test has been applied.

FIG. 8. pH-Control permits modulation of the membrane potential in primary neurons. Representative real-time traces of ASAP2s signals in primary DRG neurons co-expressing pH-Control and ASAP2s in response to 10 mM βCDA or high extracellular K+ levels (50 mM) and low Na+ levels (93 mM). Individual cell responses are from 4 independent experiments from two different batches of animals.

FIG. 9. Untargeted dsRED-stDCyD plasmid design

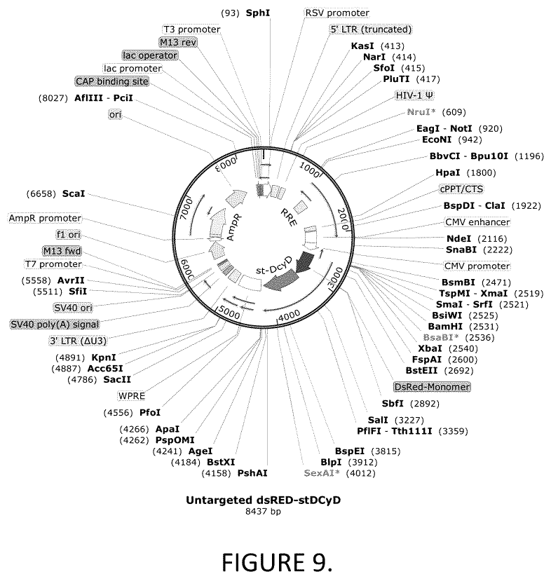

FIG. 10. mito-DsRed-stDCyD plasmid design

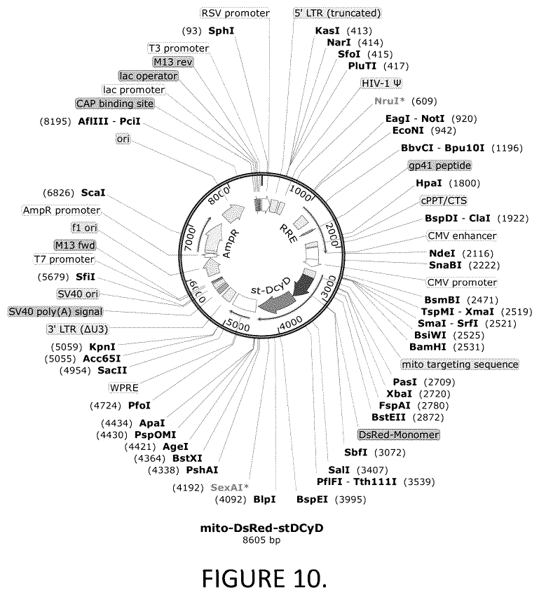

FIG. 11. DsRed-stDCyD-NES plasmid design

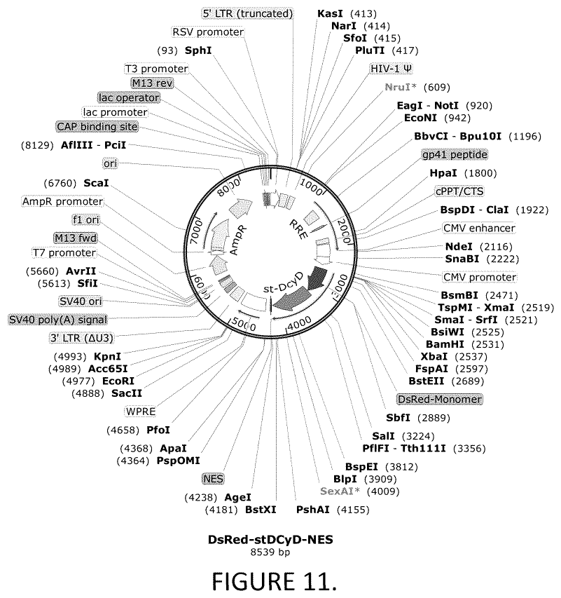

FIG. 12. DsRed-stDCyD-NLS plasmid design

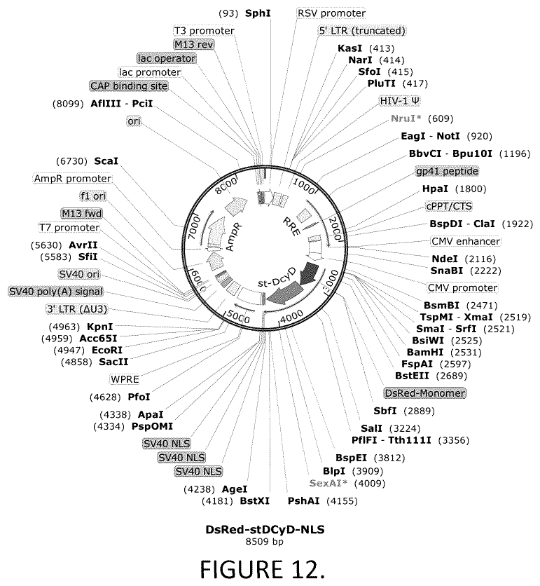

FIG. 13. Untargeted SypHer plasmid design

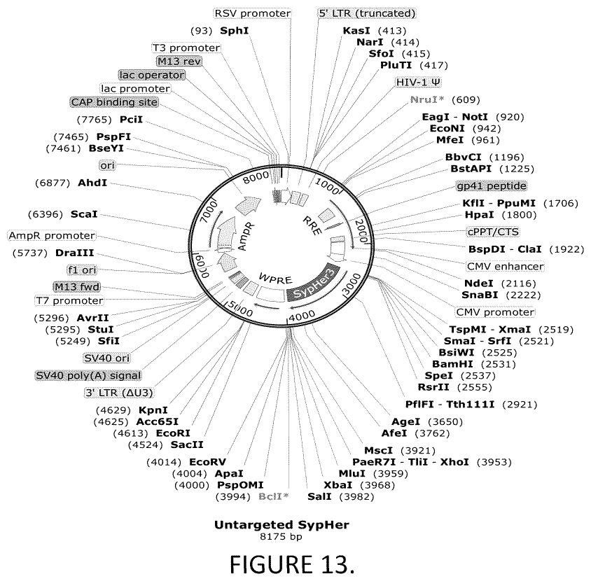

FIG. 14. mito-SypHer plasmid design

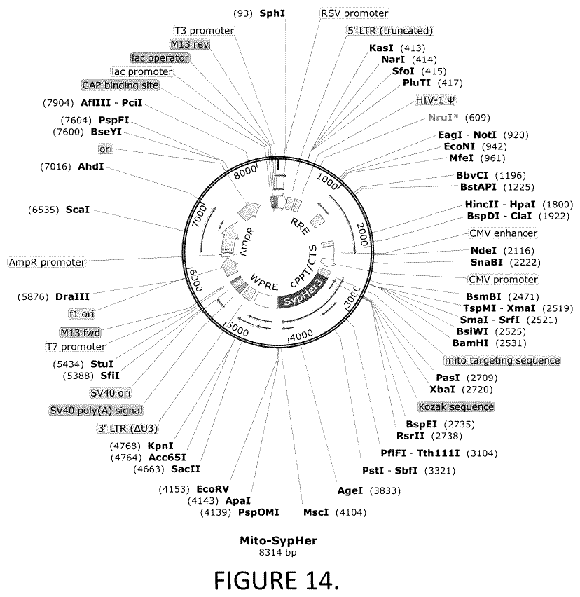

FIG. 15. SypHer-NES plasmid design

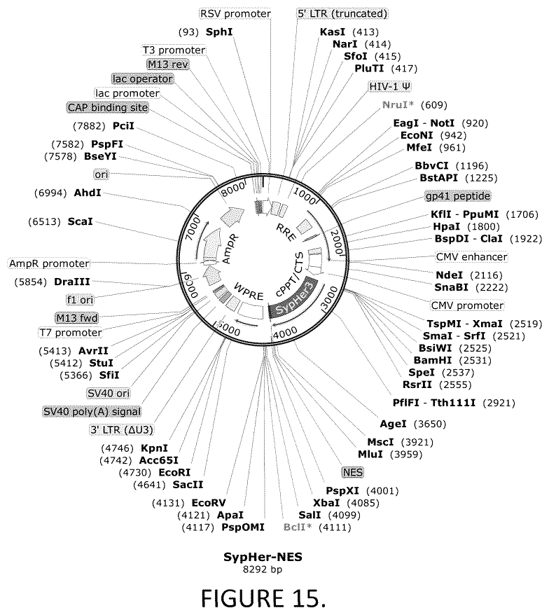

FIG. 16. SypHer-NLS plasmid design

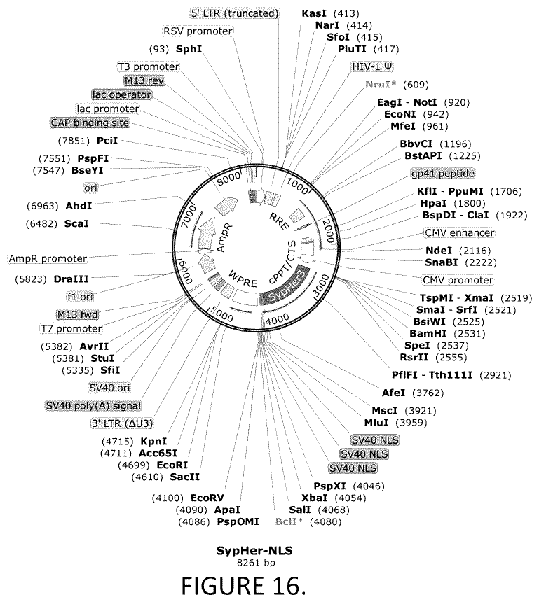

FIG. 17. dsRED-Y287FstDCyD plasmid design

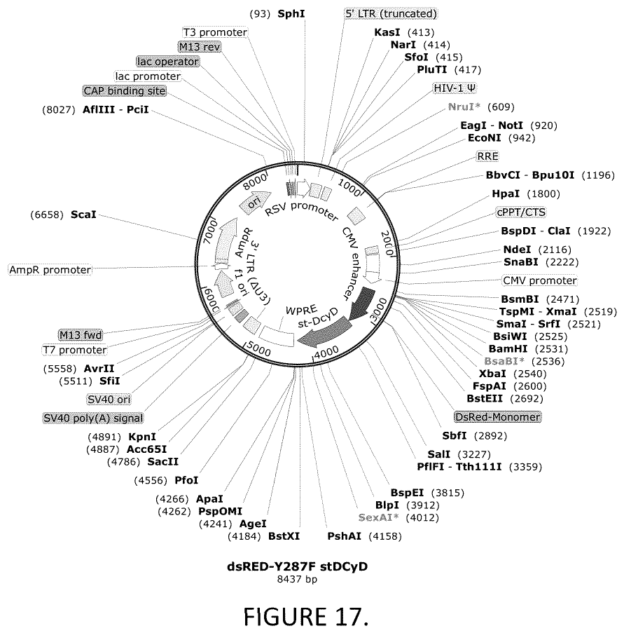

### DETAILED DESCRIPTION OF THE INVENTION

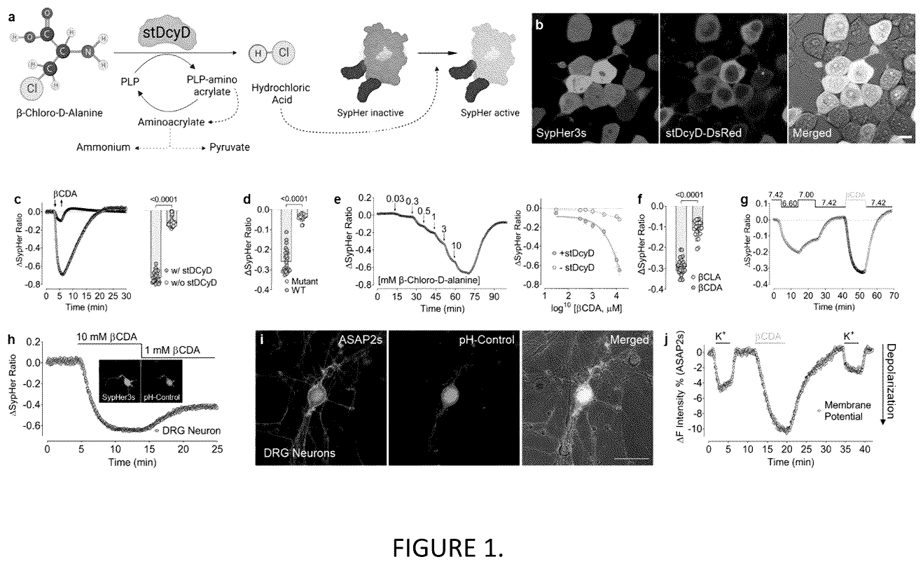

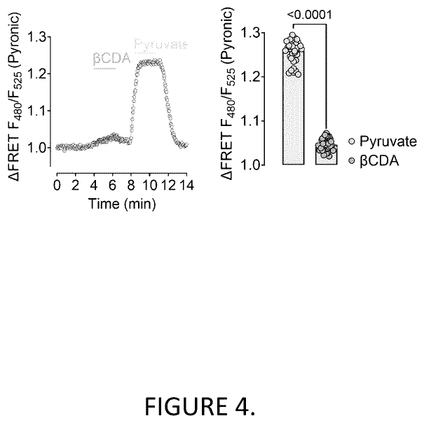

This invention presents pH-Control, an acronym for Chemogenetic Operation of iNTRacellular prOton Levels, as a novel chemogenetic approach that has been combined with the genetically encoded biosensor SypHer3s for simultaneous visualization of ultra-local acidification in living cells (FIG. 1a). This combination is important to show that for sure the chemogenetic enzyme is able to decrease the pH in the cells (by visualizing fluorescent signals from sypHer biosensor). This was a critical thing to show for the characterization of the approach. Substrate-based chemogenetic tools are recombinant proteins that are silent until their biochemical stimulus-typically an unnatural amino acid-is provided. Combined with genetically encodable biosensors, these experimental systems have opened up new lines of investigation, allowing the analysis of intracellular pathways that modulate physiological and pathological cell responses. pH-Control, which is mentioned novel chemogenetic tool in this invention, is a chimera of a red fluorescent protein variant (DsRed) and a Salmonella typhimurium-derived enzyme termed D-Cysteine Desulfhydrase (stDCyD). It has been shown in the previous studies that stDCyD converts the unnatural amino acid β-chloro-D-alanine (βCDA) to its corresponding α-ketoacid and generates the byproducts hydrochloric acid (HCl), ammonium (NH4⁺), and pyruvate in the presence of the cofactor pyridoxal 5′ phosphate (PLP). β-Chloro-D-alanine (βCDA) is a well-established antibacterial agent and cannot be metabolized by human cells and tissues. The stDCyD enzyme is differentially targetable to subcellular locales where the enzyme remains quiescent until its substrate (βCDA) is provided to generate hydrochloric acid. Theoretical calculations and experimental approaches showed that the amount of generated byproducts is neglectable (Table 1 & FIG. 4). At the same time, the change in [H⁺] equals 900% increase upon a pH change of one order of magnitude during the enzymatic activity of stDCyD (Table 1).

Table 1. Theoretical calculations of %-increase in the concentration of ions and molecules produced as (by) products of stDCyD enzyme activity upon treatment with βCDA when the pH decreases by one order of magnitude (i.e., from 7 to 6). The initial concentrations are the rough average level of ions/molecules in the human body (whether a single cell or tissue) taken from literature. The (by) products are considered to be generated equimolarly, and the average volume of cells (HEK 293T) is assumed to be 5*10-12 L.

| | Initial | | | | Percentage of |
|---|---|---|---|---|---|
| | Concentration | Final | İnitial | Final | concentration |
| Name | (M) | Concentration | Moles | Moles | alterations |
| Hydronium | 10⁻⁷ | 10⁻⁶ | 5*10⁻¹⁹ | 5*10⁻¹⁸ | 900% |
| Ion | | | | | |
| Chloride | 5*10⁻³ | 5.0009*10⁻³ | 2.5*10⁻¹⁴ | 2.50045*10⁻¹⁴ | 0.018% |
| Ion | | | | | |
| Pyruvate | 4*10⁻⁵ | 4.1*10⁻⁵ | 2*10⁻¹⁶ | 2.045*10⁻¹⁶ | 2.5% |
| Ammonia | 2*10⁻⁵ | 2.09*10⁻⁵ | 10⁻¹⁶ | 1.045*10⁻¹⁶ | 5% |
| (blood) | | | | | |

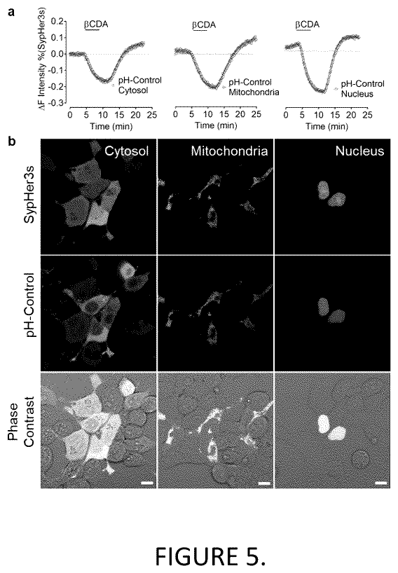

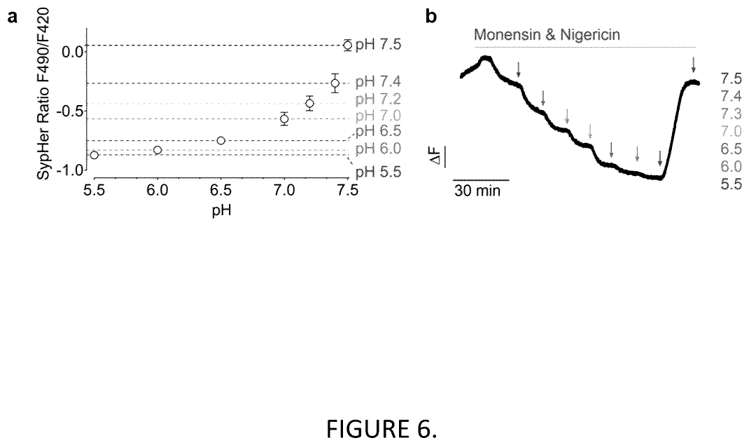

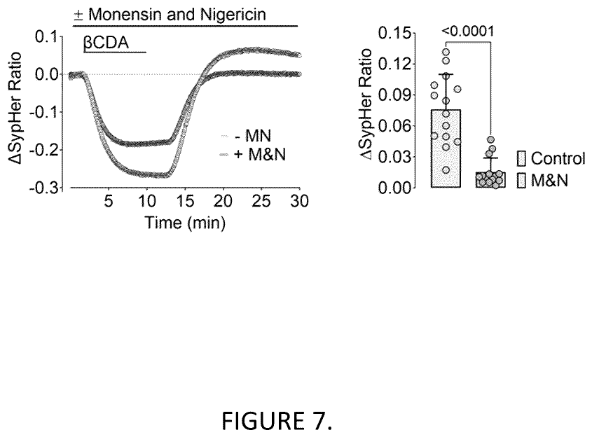

Overexpressing pH-Control with SypHer in cultured cells (HEK293T) did not show any visible toxicity (FIG. 1b) even if differentially targeted to the cytosol, mitochondria, or cell nucleus (FIG. 5). Administration of high concentrations of βCDA to cells expressing pH-Control yielded robust intracellular acidification as documented by the pH-sensitive biosensor SypHer3s. In contrast, wild-type cells without the enzyme showed marginal response to the same treatment (FIG. 1c). A single mutation at position Y287F in the stDCyD enzyme (a substitution of the tyrosin residue at position 287 of wild-type sequence of DCyD with a phenylalanin residue) yielded a dysfunctional control construct incapable of acidifying cells upon provision of βCDA (FIG. 1d). Constitutive administration of different levels of βCDA to cells expressing pH-Control showed a concentration-dependent and fully reversible SypHer response (FIG. 1e, left panel). pH-Control which is the recombinant enzyme mentioned in this invention decreases the pH in the presence of the βCDA and this drop in pH can be visualized using a pH biosensor namely SypHer. And when the SypHer's signal goes back to the base-line after removal of substrate, this shows the reversibility of the approach. At the same time cells only expressing SypHer remained unresponsive to the same treatment (FIG. 1e, right panel). Selectivity tests unveiled that the enzyme remained agnostic to the D-alanine and showed marginal responses to β-chloro-L-alanine (βCLA) in comparison to βCDA (FIG. 1f). FIG. 1g demonstrates that even low PH levels of the extracellular imaging medium are insufficient to acidify cytosolic pH to the degree to which the administration of low concentrations (1 mM) of βCDA achieved. To estimate the acidification levels using pH-Control, we calibrated the SypHer biosensor. We found the dynamic range of the pH biosensor between pH 7.5 and 5.5 (FIG. 6). Our results imply that pH-Control allows manipulation of intracellular pH in one order of magnitude, typically from pH 7.5 to 6.5 in the cytosol. Another critical observation was that after the withdrawal of βCDA, the biosensor's signal overshot the baseline after recovery, indicating a cellular alkalization, which is in line with a recent report (FIGS. 1c, 1g and FIG. 5a). To tackle this issue further, we visualized the overcorrection in cells in the presence and absence of monensin and nigericin to disentangle controlled proton transport from H⁺-channels (FIG. 7). Intracellular pH overcorrection was diminished when cells were permeabilized with monensin and nigericin.

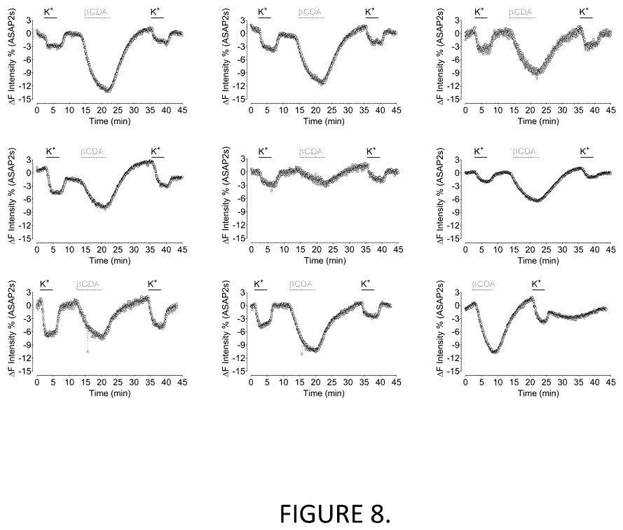

We next attempted to use the pH-Control method in mouse primary dorsal root ganglion (DRG) neurons. Cells displayed high expression levels of both constructs, pH-Control, and SypHer3s, eight days following viral transduction (FIG. 1h inset). Administration of different concentrations of βCDA yielded robust SypHer3s signals in DRG neurons (FIG. 1h). We next sought to test whether pH-Control mediated acidification is sufficient to manipulate intact primary neurons. We used the voltage sensor ASAP2s, a GFP-based biosensor that is targeted to the outer cell membrane, assuming that H⁺ generation would cause depolarization of the membrane potential (FIG. 1i). Provision of βCDA depolarized primary neurons even stronger than high extracellular potassium (FIG. 1j and FIG. 8). Overall, our results show that the pH-Control method is effective for cytosolic acidification of both cell lines and primary cells with functional consequences.

In conclusion, we developed pH-Control, a novel substrate-based chemogenetic method that enables temporal and precise manipulation of intracellular pH levels. Even in complex cell systems like neurons, pH-Control is easily combinable with any suitable biosensor for simultaneous imaging of intracellular acidification. We anticipate that introduction of our new method to transgenic animal model systems in the future will make it possible to dynamically modify pH balance in various cells and tissues alongside the ability to identify new therapeutic targets implicated in pathological acidification and physiological pathways.

This invention is unique because it exploits a non-toxic enzyme synthesized from a bacterium (Salmonella typhimurium) and can now be recombinantly generated in living cells. If co-expressed with genetically encoded biosensors, i.e., SypHer for pH measurement or hsGFP for H₂S measurement, these parameters can be precisely quantified with ultrasensitive spatio-temporal resolution. Notably, the modulation of intracellular pH and H₂S on demand in subcellular locales is not doable with any conventional approach. Since stDCyD is genetically encoded, it can be expressed in specific tissues, cell types, and subcellular locales, allowing the precise manipulation of internal pH (pHi) and H₂S levels at the desired region. In addition, fusion of stDCyD with fluorescent proteins permits tracking the localization of the enzyme. A unique element of chemogenetic approaches is the absence of activating substrate in the host body; therefore, no unspecific reaction is expected, and the amount of byproducts of the enzymatic reaction is negligible. Accordingly, in the case of stDCyD, it remains silent until the provision of βCDA or D-cysteine. In vivo, substrates can be administered to animals through drinking water, injection, or eye droplets, as shown in our previous studies with other chemogenetic enzymes. Dose-dependent response of the enzyme permits optimization of the substrate's concentration relative to the biological question.

The figure below shows the basic principle of the chemogenetic approach developed in this invention (FIG. 1a). The enzyme stDCyD can convert the unnatural amino acid beta-Chloro-D-alanine to hydrochloric acid. In the presence of a genetically encoded biosensor, this event can be measured as an optical read-out. In order to visualize the correct localization of the enzyme, it has been fused to a red fluorescent protein to generate a new recombinant chimera (FIG. 1b). Living cells subjected to the enzyme-substrate were able to acidify cellular locales. In the absence of the enzyme, only marginal pH changes were detected and documented by the pH sensor SypHer3 (FIG. 1c).

Another critical feature of this approach is that it permits the generation of HCl in cells in a concentration-dependent and reversible manner (FIG. 1e). As long as the biochemical stimulus of the enzyme is present, HCl can be produced. The pH recovers immediately within minutes to normal levels if the biochemical stimulus (substrate administration) is withdrawn from the system.

Only beta-chloro-D-alanine (βCDA) but not D-alanine can acidify cell compartments, as shown in FIG. 4A. Also, manipulation of intracellular pH levels with the relevant substrate is selective. Of note, a marginal decrease in pHi was observed by the administration of 10 mM D-Cysteine, which we believe is due to the generation of H₂S, a weak acid, as D-Cysteine is another substrate of stDCyD. Importantly, when combined with a respective biosensor (SypHer), the pH levels can be calibrated, permitting exact determination of the actual pH levels in the respective cellular locale (FIG. 6).

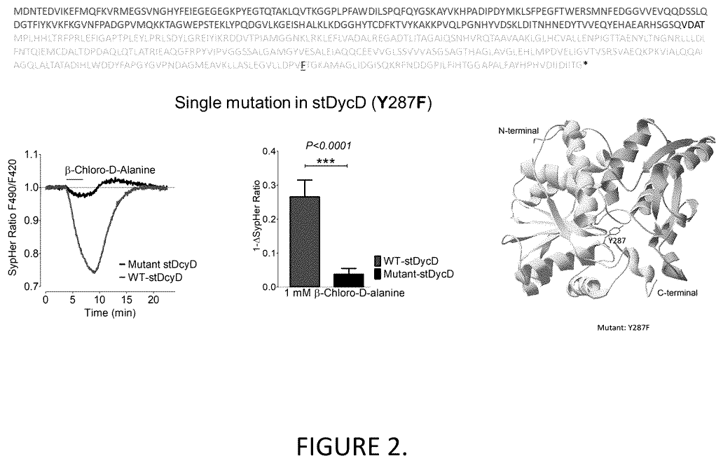

It is known from the literature that the Y287F mutation makes stDCyD inactive concerning all of its substrates. Therefore, a mutant version of the enzyme has been developed and expressed in cells to show that the mutant enzyme remains inactive in the presence of βDCA in living cells (FIG. 2).

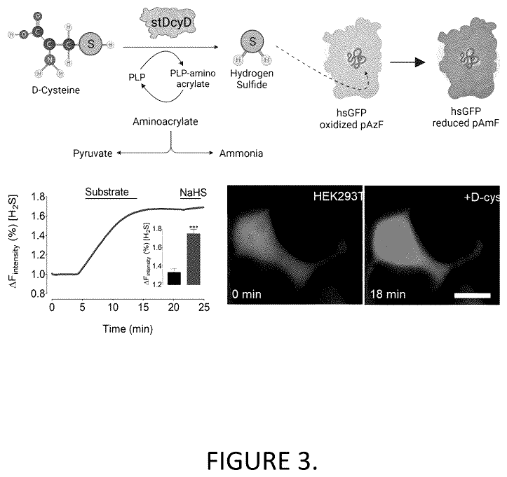

The enzyme stDCyD, which has been exploited as a novel chemogenetic approach, is multifunctional and allows the generation of H₂S if the respective substrate is provided to cells. In the presence of a suitable biosensor, i.e., hsGFP, the generation of H₂S can be selectively and in real-time detected with optical devices (FIG. 3).

### Methods

### Construct Cloning

The primers and template plasmids are listed in Table 2. All constructs are cloned into pLenti-MP2 (Addgene plasmid #36097). Cloning fragments such as targeting sequences (mito, NES, and NLS) were PCR amplified with either Taq DNA polymerase (NEB, M0273) or Q5 high-fidelity DNA polymerase (NEB, M0491S). The PCR products and cloning vectors were double digested with relevant restriction enzymes (all purchased from NEB), purified from agarose gel, and ligated using T4 DNA ligase (NEB, M0202T). The ligation products were then transformed into competent stb13 E. coli (NEB) cells. The Y287F stDcyD mutant construct was made through overlapping PCR. First, the overlapping fragments were PCR amplified using Sa1I—stDcyD-For/stDcyD-Y287F-Rev and ApaI-stop-stDcyD-Rev/stDcyD-Y287F—for primer sets. The PCR products were then used as co-templates for the next round of amplification using Sa1I-stDcyD-For/ApaI-Stop-stDCyD-Rev primers. The final product was cloned in pLenti-MP2 as previously described. All constructs were verified by whole plasmid sequencing.

### Buffers and Chemicals

Dulbecco's modified Eagle's medium (DMEM), phenol-free DMEM, penicillin and streptomycin, trypsin, and fetal bovine serum (FBS) were purchased from Pan Biotech (Aidenbach, Germany). Transfection reagent Polyjet was purchased from Signagen (Maryland, USA). β-Chloro-D-alanine was purchased from Biosynth Ltd (Compton, United Kingdom). β-Chloro-L-alanine was purchased from Medchem (Istanbul, Türkiye). All chemicals were purchased from NeoFroxx (Einhausen, Germany) unless otherwise stated. Cells outside the CO₂ incubation chamber were maintained in a storage buffer containing 2 mM CaCl₂, 5 mM KCl, 138 mM NaCl, 1 mM MgCl₂, 1 mM HEPES (Pan-Biotech, Aidenbach, Germany), 0.44 mM KH₂PO₄, 2.6 mM NaHCO₃, 0.34 mM NaH₂PO₄, 10 mM D-Glucose, 0.1% MEM Vitamins (Pan-Biotech, Aidenbach, Germany), 0.2% essential amino acids (Pan-Biotech, Aidenbach, Germany), 100 μg/mL Penicillin (Pan-Biotech, Aidenbach, Germany), and 100 U/mL Streptomycin (Pan-Biotech, Aidenbach, Germany). The pH was adjusted to 7.43 using 1 M NaOH. The cell storage buffer was sterilized using a 0.45 μm medium filter (Isolab, Germany).

For live-cell imaging experiments, a HEPES-buffered physiological solution was used consisting of 2 mM CaCl₂, 5 mM KCl, 138 mM NaCl, 1 mM MgCl₂, 10 mM HEPES, 10 mM D-Glucose, and pH was adjusted to 7.43 using 1 M NaOH. For experiments with nigericin (N7143, Merck) and monensin (M5273, Merck), imaging buffered contained 5 mM KCl, 138 mM NaCl, 1 mM MgCl₂, 20 mM HEPES, 10 mM D-Glucose, 0.2 mM EGTA and pH was adjusted to the desired value using either 1 M NaOH or 1M HCl. To prepare β-Chloro-D-alanine solutions, the powder was dissolved in imaging buffer and the pH was readjusted to 7.43.

### Cell Culture

Characterization studies have been performed in cultured human embryonic kidney cells (HEK293), grown in a high-glucose (4.5 g/L) complete medium including 10% FBS and 100 μg/ml streptomycin and 100 U/ml penicillin in a humidified incubator (37° C., 5% CO2). 24 hours before transfection, cells were seeded (˜3×105 cells per well) on a 30 mm glass coverslips No.1 (Glaswarenfabrik Karl Knecht Sondheim, Germany). At ˜70-80% confluency, cells were co-transfected with CMV-driven mammalian expression vectors (pLenti-MP2) encoding for differential targeted SypHer3s or DsRed-stDCyD enzymes using PolyJet transfection reagent according to the manufacturer's instructions. All imaging experiments were performed 24 h after transfection. HEK293T cells were cultured up to passage 30.

Primary Neuron Culture: WT C57BL/6 mice were euthanized via cervical dislocation. Bilateral DRGs from all segments were dissected and then put in an ice-cold RPMI 1640 medium (R0883, Gibco). For enzymatic dissociation, ganglia were incubated with 100 U/mL collagenases (C9407, Sigma) in a neural medium containing 2% B27 (17504-044, Gibco), 2 mM Glutamax-I (35050-61, Gibco), 100 U penicillin, and 100 mg streptomycin (15140-122, Gibco) in Neural Basal Medium (NBA, 10888-022, Gibco) at 37C, 5% CO2. Following a 40 min incubation time, the medium containing collagenase was removed, and the ganglia were washed using Hank's buffered salt solution (H9269, Sigma). The ganglia were further enzymatically dissociated using 1 mg/mL trypsin (25300-054, Gibco) in the neural medium for 15 min at 37° C., 5% CO2. At the end of incubation, 50 mg/mL DNAse (D4513, Sigma) was added to the trypsin solution cell suspension to inhibit free DNA fragments, and the tissues were triturated by pipetting to obtain single cells. 30 min after incubation at 37° C., 5% CO2, the cell suspension was spun at 120 g for 3 min and resuspended neural medium supplemented with 10% fetal calf serum and 700 mg/mL trypsin inhibitor (T6522, Sigma) to inhibit enzymatic activity. To purify DRG neurons from the satellite cells and cell debris, the cell suspension was carefully put into 10%, 35%, and 60% percoll (P4937, Sigma) gradients and spun at 300 g for 20 min. The total sensory neurons were collected from approximately 35% percoll layer, and the cell suspension was spun at 120 g for 3 min. The pellet was resuspended with a neural medium without antibiotics for virus transfection. Following that procedure, cells were seeded onto a petri dish coated with 10 mg/ml laminin (L2020, Sigma). One day after incubation, cells were transduced with lentivirus, as described above.

### High Titer Virus Purification

HEK293T cells were transfected with helpers MDL, RSV-rev, VSVG (gifts from Didier Trono, Addgene plasmids #12251, #12253, and a gift from Arthur Nienhuis & Patrick Salmon, Addgene plasmid #35616,) together with transfer plasmids (pLenti-MP2-SypHer3s or pLenti-MP2-DsRed-stDCyD) with 1:4 plasmid: PEI ratio when reached to 70% confluency. Lentivirus-containing medium was harvested at 72 h of transfection followed by centrifugation at 3000 g for 3 min and filtered through 0.45 ρm PES filter (SLHP033RS, Millipore). Lentiviral particles were purified via centrifugation at 10,000 g for 4 h at 4° C. on a 20% sucrose cushion and further concentrated with the same centrifugation parameters in ice-cold 1x PBS.

AAV-HEK293cells at 70% confluency were transfected with pAAV-hSyn-ASAP2s, pAdDeltaF6, and pAAV2/1 (a gift from Francois St-Pierre, and gifts from James M. Wilson, plasmids were purchased from Addgene plasmids #112867, #112867, #101276) with 1:4 plasmid:PEI ratio. Cells were collected with a cell scraper 72 h after transfection and pelleted by centrifugation at 300 g for 5 min. Cell pellets were resuspended in lysis buffer (150 mM NaCl, 20 mM Tris, 1 mM MgCl₂, pH: 8) and lysed following a 3x freeze-thaw cycle and subsequent sonication and Benzonase (E8263, Sigma) treatment for 45 min at 37° C. Cell debris was removed with centrifugation at 300 g for 20 min at 4° C. AAV containing supernatant was ultracentrifuged at 220.000 g through Iodixanol (D1556, Sigma) gradient (60%, 40%, 25%, 17%) for further purification for 2 h. AAV particles were collected from the interface between gradients of 40%-60%. Iodixanol was removed, and AAV particles were concentrated in a storage buffer (1x PBS, 5% D-sorbitol, 200 mM NaCl) using 100K columns (UFC910024, Millipore) with 3x centrifugation at 4° C. and 300 g for 30 min.

### Live Cell Imaging

Widefield imaging experiments were performed on a Zeiss Axio Observer.Z1/7 (Carl Zeiss AG, Oberkochen, Germany) equipped with an LED light source Colibri 7 (423/44 nm, 469/38 nm, 555/30), PlanApochromat 20x/0.8 dry objective, Plan-Apochromat 40x/1.4 oil immersion objective, a monochrome CCD camera Axiocam 503. A custom-made pump-driven perfusion system was used to administrate and withdraw substrates to cells placed in a metal perfusion chamber (NGFI, Graz, Austria). SypHer signals were imaged by alternately exciting cells using a motorized dual-filter wheel equipped with beam splitters (FT455 (for SypHer low, F420) and FT495 (for SypHer high, F490)). Emissions were alternately collected using a bandpass filter (BP 525/50). DsRed-stDyCD emission was collected using the filter combinations FT570 (BS) and emission filter 605/70. During live-imaging data acquisition was performed using Zen Blue 3.1 Pro software (Carl Zeiss AG, Oberkochen, Germany). Confocal imaging was performed using a laser scanning confocal microscope LSM 800 (Zeiss, Germany) equipped with a Plan-Apochromat 40x/1.3 DIC (UV) VIS-IS oil immersion objective. SypHer biosensors were excited with a 488 nm and 405 nm laser, and emissions were collected using a 509 nm filter system. SypHer signals were acquired with an A GaAsP-PMT detector and 400-565 nm filter using a Multialkali-PMT detector. DsRed-stDCyD constructs were excited using a 561 nm laser, and the emission wavelength was captured between 616 and 700 nm. The digital detector gain for all channels was set at 1; detector gain was applied between 500 and 1000 V. Laser intensities were set between 0.90% and 0.95%, and the pinhole was set between 29 and 32 μm according to the expression level of the fluorescent proteins. Bright-field mode was imaged using a photodiode detector. Zen Blue 3.1 software (Zeiss, Germany) was used to determine regions of interest.

### Statistical Analysis

Image analysis was performed using GraphPad Prism software (GraphPad Software, San Diego, CA, USA). All experiments were repeated at least in triplicates, and the exact number of experiments is given as ‘N’, and the total number of cells imaged is indicated as ‘n.’ in the figure legends. For instance: 3/32 indicates N=3 (triplicate cultures) and n=32 (number of cells imaged in this experiment). Statistical comparison of the two groups was evaluated using a two-tailed Student's t-test.

The homeostasis of pHi and pHe play critical roles in neuronal burst firing, silencing, and neurotransmission, therefore, in the physiology and pathophysiology of the central nervous system. It seems that the correlation between pHi and neuronal excitability depends on the type of neurons in a way that, for example, chemosensitive neurons show higher levels of excitatory responses upon intracellular acidosis when compared to hippocampal neurons that exhibit milder responses. To evaluate how changes in pHi affect the chemosensitive response of locus coeruleus neurons, Filosa et al., applied hypercapnic acidosis, isocapnic acidosis, isohydric hypercapnia, acidified Hepes-buffered medium, or propionate-containing HCO₃− buffer and then measured the pHi alteration and neuronal firing rate related to that change. This way, they demonstrated that the acidic pHi challenge, at least in part, is responsible for a rise in firing rate in locus coeruleus neurons. However, the exact sequential mechanisms and pathways remained elusive. This difficulty is mainly due to the lack of a robust and ubiquitous approach for managing the intracellular pH level in a reliable, controlled, and physiological manner.

On the other hand, more than 60 genes encode genes directly responsible for pHi regulation through the cell membrane, whether directly or indirectly. Therefore, it seems that regulating the pHi by manipulating the endogenous proton regulation machinery within the cells would not be reasonable, and in most cases, it backfires. More importantly, the proton level is not independent of other ions, and molecules in the intracellular matrix, and changing the pH through manipulating pH regulatory elements affects the bioavailability of different molecules and ions (e.g., Na⁺, K⁺, and Cl⁻), namely counterions' perturbation, and all the following data provided this way would be doubtful.

On the contrary, chemogenetic tool mentioned in this invention can tackle this matter with the highest accuracy and efficacy. The stDCyD enzyme remains dormant until supplementation of its substrate, which is provided exogenously and can be stopped at any time. Therefore, it adds a layer of specificity and highly increases the spatiotemporal resolution of the chemogenetic approach.

In addition to unraveling how pH alteration affects cell behavior in general and at the macro level, such as cell migration and proliferation, the impact of pH on protein conformation and function in the physiological context is significant. Some proteins harbor ultrasensitive switches and show striking conformational and functional alterations by a bit of change in pH in vitro. However, the only way to solidly monitor this phenomenon is the intracellular regulation of pH level in a highly controlled manner; otherwise, the pH alterations may be subtle or harsh and not provide physiologically-relevant data. In opposition to studies that tried to unravel the role of pH level in cellular signaling using conventional methods in vitro, this innovative chemogenetic tool opens promising windows in investigating the downstream pH-dependant signaling pathways in a cellular setting with high spatiotemporal resolution.

MDNTEDVIKEFMQFKVRMEGSVNGHYFEIEGEGEGKPYEGTQTAKLQVTK
GGPLPFAWDILSPQFQYGSKAYVKHPADIPDYMKLSFPEGFTWERSMNFE
DGGVVEVQQDSSLQDGTFIYKVKFKGVNFPADGPVMQKKTAGWEPSTEKL
YPQDGVLKGEISHALKLKDGGHYTCDFKTVYKAKKPVQLPGNHYVDSKLD
ITNHNEDYTVVEQYEHAEARHSGSQVDATMPLHHLTRFPRLEFIGAPTPL
EYLPRLSDYLGREIYIKRDDVTPIAMGGNKLRKLEFLVADALREGADTLI
TAGAIQSNHVRQTAAVAAKLGLHCVALLENPIGTTAENYLTNGNALLLDL
ENTQJEMCDALTDPDAQLQTLATRIEAQGFRPYVIPVGGSSALGAMGYVE
SALEIAQQCEEVVGLSSVVVASGSAGTHAGLAVGLEHLMPDVELIGVTVS
RSVAEQKPKVIALQQAIAGQLALTATADIHLWDDYFAPGYGVPNDAGMEA
VKLLASLEGVLLDPVFTGKAMAGLIDGISQKRFNDDGPILFIHTGGAPAL
FAYHPHVDIIDIITG*

The primary sequence of the tool mentioned in the invention is shown above. The boldly highlighted sequence represents the primary sequence of the red fluorescent protein DsRed, and the black highlighted sequence is the primary sequence of the Salmonella typhimurium-derived enzyme termed stDCyD. Single mutation has been introduced to position 287 as indicated to yield a nonfunctional enzyme (as shown underlined).

The primers and the template plasmids are shown in Table 2 and the intended uses of these designed plasmids are listed below;
Untargeted dsRED-stDCyD: to change the pH in whole cell, without specific targeting,
mito-DsRed-stDCyD: to change the pH only in the matrix of the mitochondria,
DsRed-stDCyD-NES: to change the pH only in cytosol,
DsRed-stDCyD-NLS: to change the pH only in the nucleus,
Untargeted SypHer: to visualize the pH changes in the whole cells without specific targeting,
mito-SypHer: to visualize the pH changes only in the mitochondria,
SypHer-NES: to visualize the pH changes in the cytosol,
SypHer-NLS: to visualize the pH changes in the nucleus,
dsRED-Y287F stDCyD: to show that the pH changes that observed is only because of the function of the enzyme and if the enzyme is made non-functional with Y287F mutation, the pH cannot change anymore.

TABLE 2
The primers and template plasmids

| Plasmid | | |
|---|---|---|
| Name | Forward Primer | Reverse Primer |
| Untargeted | BamHI-DsRed-stD-FWD | ApaI-stop-AgeI-stD-dsRed-REV |
| dsRED- | ATAATAGGATCCTCTAGAGC | TATTATGGGCCCTTAACCGGTTATGATA |
| stDCyD | CACCATGGACAACACCG | TCTATGATATCAACGTGGG |
| mito-DsRed- | Mito-stD-BamHI-FWD | Mito-stD-XbaI-REV |
| stDCyD | TATGGATCCGAATTCGCCAC | ATATCTAGACTTGGCCCTGGGGACTG |
| | CATGTCTGTTC | |
| DsRed- | AgeI-stD-NES-FWD | stD-NES-ApaI-REV |
| stDCyD-NES | TATACCGGTCTGCCCCCCCT | ATAGGGCCCGTGGCAACTTCCAG |
| | GGAG | |
| DsRed- | stD-AgeI-NLS-FWD | stD-NLS-ApaI-REV |
| stDCyD-NLS | CAAGTTCTGCTTCTGACCGA | ATAGGGCCCTTCTAGAGGCTCGAG |
| | GGGC | |
| Untargeted | BamHI-UN.SypHer-pLenti-FWD | XbaI-UN.SypHer-pLenti-REV |
| SypHer | TATGGATCCACTAGTGCCACCATGTC | GATATCTAGATTAACGCGTCTCGAG |
| | CGGAC | AACCGCCTGTTTTAAAAC |
| mito-SypHer | XbaI-SypHer-mito-pLenti-FWD | ApaI-SypHer-mito-pLenti-REV |
| | TATTCTAGAGCCACCATGTCCGGACCG | GCAATAGGGCCCTTAAACCGCCTGTTT |
| | CTG | TAAAACTTTATCG |
| SypHer-NES | MluI-SypHer-NES-pLenti-FWD | XbaI-SypHer-NES-pLenti-REV |
| | TATACGCGTCTGCCCCCCCTGGAG | ATATCTAGAGTGGCAACTTCCAGGGCCAG |
| SypHer-NLS | MluI-SypHer-NLS-pLenti-FWD | stD-NLS-ApaI-REV |
| | GTATACGCGTACCGGTGATCCAAAAA | ATAGGGCCCTTCTAGAGGCTCGAG |
| | AG | |
| dsRED-Y287F | SalI-stDcyD-For | ApaI-stop-stDcyD-Rev |
| stDCyD | CAGGTCGACGCCACCATG | TATCTGTACAGGGCCCTATGAATTC |
| | stDcyD-Y287F-For | stDcyD-Y287F-Rev |
| | CGATCCAGTTTTCACTGGGAAGG | GCCTTCCCAGTGAAAACTGGATCG |

1. In vitro use of a recombinant D-Cysteine Desulfhydrase (DCyD) enzyme as a chemogenetic tool for manipulation of internal pH (pHi).

2. In vitro use of a recombinant D-Cysteine Desulfhydrase (DCyD) enzyme of claim 1, pH-dependent signaling and/or metabolic pathways.

3. In vitro use of a recombinant D-Cysteine Desulfhydrase (DCyD) enzyme of claim 1, wherein DCyD enzyme is derived from Salmonella typhimurium.

4. A chemogenetic method of modulating the internal pH, the method comprising:
i. providing sequence of D-Cysteine Desulfhydrase (DCyD) enzyme,
ii. cloning the sequence of mentioned enzyme to cloning vector,
iii. transfer of the cloning vector into the living cells whose pH is desired to be changed,
iv. administration of the substrates of the enzyme to the cells where the recombinant enzyme is produced.

5. A chemogenetic method of claim 4, the method comprising: cloning at least one pH-sensitive fluorescent protein sequence to the cloning vector.

6. A chemogenetic method of claim 4, the method comprising: transferring vector comprising at least one pH biosensor sequence into the living cells.

7. A chemogenetic method of claim 4, wherein the substrates of the enzyme are β-Chloro-D-alanine (βCDA) or D-cysteine.
